(19) United States
(12) Patent Application Publication
(10) Pub. No.: US 2013/0089576 A1
(43) Pub. Date: Apr. 11, 2013

(54) IMPLANTABLE EMBOLIC SCAFFOLDS THAT PROMOTE HEALING

(71) Applicants: Lawrence Livermore National Security, LLC, Livermore, CA (US); The Texas A&M University System, College Station, TX (US)

(72) Inventors: Duncan J. Maitland, College Station, TX (US); Thomas S. Wilson, San Leandro, CA (US)

(73) Assignees: The Texas A&M University System, College Station, TX (US); Lawrence Livermore National Security, LLC, Livermore, CA (US)

(21) Appl. No.: 13/633,388

(22) Filed: Oct. 2, 2012

Related U.S. Application Data
(60) Provisional application No. 61/543,146, filed on Oct. 4, 2011.

(51) Int. Cl. A61L 27/18; A61L 27/56

(52) U.S. Cl. CPC A61L 27/18; A61L 27/56
USPC 424/400; 424/78.37; 521/163

(57) ABSTRACT

Implant devices and structures that reduce inflammation and promote healing of the area of implant. Specifically, the use of shape memory open cell biocompatible polymer foams for implants that assist in and promote healing and especially in filling and sealing aneurisms.

### CROSS-REFERENCE TO RELATED APPLICATIONS

This application claims benefit under 35 U.S.C. §119(e) of U.S. Provisional Patent Application No. 61/543,146 filed Oct. 4, 2011 entitled “Implantable Embolic Scaffolds That Promote Healing,” the disclosure of which is hereby incorporated by reference in its entirety for all purposes. This invention was made with government support under grant from the National Institutes of Health and the government may have rights to this invention.

### STATEMENT AS TO RIGHTS TO INVENTIONS MADE UNDER FEDERALLY SPONSORED RESEARCH AND DEVELOPMENT

The United States Government has rights in this invention pursuant to Contract No. DE-AC52-07NA27344 between the United Stated Department of Energy and Lawrence Livermore National Security, LLC for the operation of Lawrence Livermore National Laboratory.

### BACKGROUND

1. Field of Invention

Implant devices and structures that reduce inflammation and promote healing of the area of implant. Specifically the use of shape memory open cell biocompatible polymer foams for implants that assist in and promote healing.

2. Background

Cerebral aneurysms are geometric abnormalities or a bulging of a segment of an artery within the vasculature of the brain. Aneurysms are at risk of rupturing and within the United States one in fifty people have an unruptured aneurysm. Upon rupture of one of these aneurysms, a subarachnoid hemorrhage (SAH) is said to have occurred and can result in a fatal or severely debilitating event. SAH affect approximately thirty thousand people per year in the United States.

Current treatments for these arterial abnormalities include methods of isolation from the normal vasculature, and subsequent stabilization of the vulnerable portion of the artery. Isolation methods involve surgical clipping, or filling of the aneurysm dome, thereby preventing subsequent rupture. Surgical clipping, although highly effective for treating aneurysms, involves invasive surgery in the form of a craniotomy. Additionally, for patients that are not viable candidates for surgery or for patients where the location of their aneurysm is in an area of the brain that are considered risky, i.e. the base of the brain, surgery is not a viable or preferred option.

Filling methods involve endovascular navigation to the site of the aneurysm with the aid of fluoroscopy utilizing an injected contrast agent for positioning of the devices. Aneurysm filling methods involve isolation of the weakened portion of the aneurysm from the constant impingement of arterial blood flow by filling the aneurysm sac with a material that promotes acute clotting and chronic healing at in the aneurysm. For optimal treatment via filling methods, the filling material would become incorporated into the aneurysm sac, and the interface of the filling material would become walled off by the endothelial cell layer of the parent artery, also known as the neointima. This encasing of the filler essentially walls off the sac from the parent vessel, permanently stabilizing the weakened portion of the artery and preventing subsequent rupture.

Current aneurysm filling devices are composed of different filling materials. These materials include bare metal coils, including platinum, such as Guglielmi Detachable Coils (GDC), (MicroVention, Terumo, Tustin, Calif.), hydrogel coated coils (MicroVention, Terumo, Tustin, Calif.), polymer enhanced coils (Stryker, Kalamazoo, Mich.), (Micrus, Raynham, Mass.) and (Johnson and Johnson, New Brunswick, N.J.) or in situ polymerized materials, such as Onyx® liquid embolic materials (EV3, Irvine, Calif.) and (Covidien, Mansfield, Mass.). These devices have proven to be clinically successful at filling small necked aneurysms (<4 mm in DIA). However, these devices could be improved upon with respect to the biological activity and safety of delivery for these implanted materials.

It has been demonstrated by Murayama et al. and Szikora et al., in 2001 and 2006 respectively, that GDCs can be a continual source of inflammation within the aneurysm after implantation (Murayama Y, Vinuela F, Tateshima S, Song J K, Gonzalez N R, Wallace M P. Bioabsorbable polymeric material coils for embolization of intracranial aneurysms: a preliminary experimental study. J Neurosurg 2001 March; 94(3):454-463). Szikora et al. reported that after multiple years, GDCs are still not completely endothelialized at the aneurysm/parent artery interface and the aneurysm is not optimally stabilized (Szikora I, Seifert P, Hanzely Z, Kulcsar Z, Berentei Z, Marosfoi M, et al. Histopathologic evaluation of aneurysms treated with Guglielmi detachable coils or matrix detachable microcoils. AJNR Am J Neuroradiol 2006 February; 27(2):283-288).

Additionally, Onyx undergoes polymerization in situ. Delivery of Onyx involves the placement of a balloon catheter over the parent artery while the polymer is injected into the fundus via a catheter. This delivery technique does not guarantee a seal at the balloon artery interface, and therefore, is prone to occlude distal arteries.

Beyond a lack of biological activity and safety of delivery, GDC coils also have a tendency to have low volume filling, compact over time, which could result in reformation of side wall aneurysms adjacent to the original aneurysm. Other efficacy issues surrounding GDCs involve recanalization, migration of coils into the parent vessel, inability to treat wide necked aneurysms and potential rupture of the filled aneurysm. Both recanalization and rupture of the aneurysm would require further treatment to stabilize the patient.

Shape memory polymers (SMP) have been proposed as alternative filling methods for treatment of cerebral aneurysms (See, e.g Ortega J, Maitland D, Wilson T, Tsai W, Savas O, Saloner D. Vascular dynamics of a shape memory polymer foam aneurysm treatment technique. ANN BIOMED ENG 2007; 35(11):1870-1884). Polymer coated GDC coils have also been suggested as filling materials and have been shown to have favorable results in vivo, suggesting that polymeric materials could be alternative materials for treating aneurysms with better long term healing. It has also been shown that polyurethane foams have favorable biocompatibility in vivo for larger aortic aneurysm animal models. The present invention focuses on the biocompatibility of polyurethane based SMP foams as filling material for the treatment of intracranial aneurysms.

SMPs are materials that have the ability to be made into a primary shape, and upon an increase in the bulk temperature of the material above its transition temperature, can be deformed and programmed into a temporary shape. Programming of these materials occurs when the temperature of the material is brought below its transition temperature, while keeping the deformation force constant. A device made from these materials will remain in this programmed shape until it had encountered an additional stimulus that would raise the material's temperature above its transition temperature, at which point it would recover to its primary shape. This ability to change via the application of a stimulus from one shape to another has made these materials attractive to many medical device developers. These polymers possess characteristics similar other polymeric materials; they are capable of being molded or foamed into an open celled architecture and have the potential to be incorporated into medical devices. These characteristics are utilized in the present invention.

The same considerations in treating aneurisms apply to similar treatments of other vascular and septal abnormalities.

### SUMMARY

Implanted devices and structures into living tissue frequently results in inflammation and impeded healing at the site of the implant. This invention is, in one aspect, a method of reducing inflammation and of promoting healing at implant sites by the use of open cell polymer foams. In another aspect it is a composition of such open cell polymer foams that promote healing.

### BRIEF DESCRIPTION OF THE DRAWINGS

FIG. 1A and FIG. 1B provide a representation of Suture interaction from a test of an embodiment of the invention.

FIG. 2A and FIG. 2B provide a representation of pathology scoring of neovascularization throughout an aneurysm dome at zero, thirty and ninety days from a test of an embodiment of the invention.

### DETAILED DESCRIPTION OF SPECIFIC EMBODIMENTS

This invention is, in broad aspect, porous (partially or fully open celled) foam scaffolds that, when implanted in the body, reduce inflammation and promote healing. For vascular embolic applications, the implantation of the foam scaffold results in a sequential series of healing responses: localized, contained blood clot; minimal inflammation; breakdown and removal of fibrin throughout the scaffold; ingrowth of extracellular matrix (e.g. collagen) throughout the scaffold; vascular ingrowth throughout the scaffold.

The healing response of the scaffold is a function of material and structure of the scaffold. Open celled foams with cell sizes ranging from 50 nm through several (about 5) mm are used to construct the scaffold. The reticulation of the cells (removal of membranes between cells) should be at least 20% and preferably at least 50% or more of the membranes removed or partially open. Cell size distribution can be broadly or narrowly distributed. Suitable materials for construction are biocompatible polymers, including amorphous crosslinked polyurethane, polyacrylates, polymethacrylates, polyolefins, polyorganosiloxanes, polyesters, polyethers, and copolymers of these families. Foam density should be 0.1 g/cm³ or lower.

Early after implant, the foam scaffold of the invention promotes clots within the scaffold pores/cells. These clots form an inter-connected framework that promotes transport and movement of healing throughout the scaffold. The ability for biological healing response transport to move throughout the scaffold is key to the superior healing of the scaffolds. In contrast, current embolic coils, with or without hydrogel coatings, require the formation of large volume clots to form and fill around the coils. These large clots resist healing.

One of the important embodiments of the invention is polyurethane based shape memory polymer (SMP) foam for the treatment of, inter alia, intracranial aneurysms. These foams have an expansion force less than the amount necessary to rupture an aneurysm (or other vascular abnormality), have been demonstrated in a pilot study to be biocompatible in vitro for neat materials and low density foams. To verify and further demonstrate biocompatibility in vivo, these foams were implanted in a porcine vein pouch animal model for zero, thirty and ninety days. Gross evaluation of healing, low vacuum scanning electron microscopy (LV-SEM) and histology was performed verifying the healing response exhibited by the implanted foams.

The same results can be obtained with other vascular and septal implants where blood clotting is an important aspect of healing. The methods and compositions of this invention are equally applicable to human and animal treatments.

Foam materials of the invention were compared to U.S. Food and Drug Administration (FDA) approved silk and polypropylene sutures as reference for the level of inflammation elicited by the SMP foams of the invention. The polyurethane SMP foam useful for the treatment of aneurysms has been shown to be an effective filler material that did not exhibit excessive inflammation and did promote healing throughout the aneurysm. As described in detail below, healing progressed from the perimeter to the core by ninety days of implantation. Because of the limited amount of inflammation promoted by this polymer material it clearly is a viable and valuable material for any number of blood contacting or implanted devices.

The following examples demonstrate the fabrication techniques used to prepare and test polyurethane SMPs useful for the open cell foams of this invention.

SMP Fabrication

Exemplary aneurism filling devices according to the invention were fabricated out of Polyurethane SMP foams based on chemistry reported by Singhal et al. in 2012. (Singhal P, Rodriguez J N, Small W, Eagleston S, Van de Water J, Maitland D J, et al. Ultra Low Density and Highly Crosslinked Biocompatible Shape Memory Polymer Foams. Journal of Polymer Science, Part B: Polymer Physics 2012). Monomers of N,N,N′,N′-145 Tetrakis(2-hydroxypropyl)ethylenediamine (HPED) (Sigma-Aldrich, St. Louis, Mo.), 2,2′,2″-Nitrilotriethanol (TEA) (Sigma-Aldrich, St. Louis, Mo.) and 1,6 Diisocyanatohexane (HDI) (TCI America, Portland, Oreg.) were used along with both chemical and physical blowing agents. Foams were made by preparing a pre-polymer. Hydroxy Propyl Ethylene Diamine (HPED), Tri-Ethanol Amine (TEA) and Hexamethylene Di-is 150 ocyanate (HDI) were combined and mixed thoroughly via vortex mixing until a single phase was formed. The pre-polymer was allowed to cure at room temperature for two days prior to foaming.

The pre-polymer was added to an additional amount of TEA, catalysts and surfactants and mixed vigorously. The rising polymer mixture was then put in the oven at 90° C. and allowed to continue to foam. The foam was allowed to cure for twenty minutes in the oven and then placed at room temperature for at least two days.

Device Fabrication

Exemplary spherical SMP foam devices were fabricated using a scalpel into dimensions of approximately 8 to 12 mm in diameter. Varying sizes of SMP foam devices were fabricated to tailor the needs of the vascular surgeon upon implantation.

Removal of surfactants and catalysts from the devices were achieved by cleaning via 0.1 M HCl and detergent, Contrad® 70 (Decon Laboratories, Inc., King of Prussia, Pa.) solution. The devices were initially placed in glass vials and placed in a sonication bath while submerged in 0.1N HCl for two hours. This step was followed by changing the solution within the vial to 80:20 volume %, deionized water-Contrad® 70 solution, and then placed into the sonication bathed for fifteen minutes. The detergent solution was removed with multiple washes of deionized water; it was determined that there was no more detergent when there were no visible bubbles upon shaking. After the samples were free of detergent, the samples were placed back into the sonication bath for another fifteen minutes within a vial of deionized water. These steps were repeated and the samples were dried in an oven over night at 50° C. The samples were visually examined for loose struts on the perimeter via magnification.

Device samples with loose struts were removed via trimming. The samples were trimmed using a scalpel and calipers to restore the spherical shape if they were misshapen during the cleaning process. Each device sample was individually sealed in a sterilization pouch and sterilized by Ethylene oxide (EtO), and allowed to degas for forty eight hours prior to implantation.

Porcine Animal Model

All animal experiments were conducted in accordance with policies set by the Texas A&M University's Institutional Animal Care and Use Committees (IACUC), and meet all federal requirements, as defined in the Animal Welfare Act (AWA), the Public Health Service Policy (PHS), and the Humane Care and Use of Laboratory Animals. Additionally, NIH guidelines 192 (or for non-U.S. residents similar national regulations) for the care and use of laboratory animals (NIH Publication #85-23 Rev. 1985) were observed. A porcine aneurysm model was utilized in these studies, detailed by Guglielmi et al. in 1994 (Guglielmi G, Ji C, Massoud T F, Kurata A, Lownie S P, 708 F. Vinuela F, et al. Experimental saccular aneurysms Neuroradiology 1994; 36 (7):547-550.) This model involves removal of a section of the external jugular vein which is cut into two segments. An end of one of segments is then sewed to aneurysm site of one of the common carotid arteries. Previously an ellipsoid section had been removed from the carotid artery, which would then make up the neck diameter. The donor vein segments that had been sewn on from an end had been closed at the other end to create the apex of the aneurysm with a suture. This technique was repeated on the other carotid artery with the remaining donor segment, thereby creating two berry shaped aneurysms within the neck of the pig. Suture and aneurysm stability between the parent vessel and the graft were tested on both aneurysms via angiography. This was done to visualize the integrity of the aneurysm for leaks and the ability to be filled with blood. The SMP devices were placed in each of the aneurysms by reopening the top suture and reclosing after proper placement had been achieved. Device filling and aneurysm integrity were tested on both aneurysms via angiography after each one had been implanted.

Implantation (zero, thirty and ninety days) Devices were implanted into each of the carotid aneurysms after soaking in 37° C. saline, and allowed to remain in place for either zero, thirty or ninety days. After the zero, thirty or ninety day period had been completed, the animals were sacrificed. Each of the aneurysms and their parent vessels were preserved with formalin at the point of isolation. The aneurysms were then removed for gross and histological evaluation.

Gross Evaluation of Healing

Each aneurysm was isolated post-mortem and removed from the rest of the vasculature. The parent vessels were bisected parallel to the flow of the parent vessel, and each aneurysm was observed at the main artery and graft interface for endothelialization of the lesion en face.

Microscopic Evaluation of Healing

To determine the overall healing and stabilization throughout the device and the vessels, LV-SEM imaging was used to determine completeness of endothelialization of the parent vessel and device interface and pathology was used to determine the amount of healing and inflammation that occurred within the dome of the aneurysms. Multiple stains were utilized in the pathological evaluation of the harvested vessels.

LV-SEM: Endothelialization

Healing of the aneurysms was evaluated for multiple characteristics; one of those characteristics included evaluation of the amount of endothelialization at the device and parent vessel interface. Evaluation of the amount of endothelialization is a form of measuring the restoration of the neointima of the parent artery at the device interface. Presence of neointima and progression of the covering was tracked over time via scanning electron microscopy (LV-SEM). Aneurysms were imaged via LV-SEM for a microscopic evaluation of the progression of endothelialization at the aneurysm lesion site at each time point.

Pathology:

Multiple stains were used to evaluate the amount of healing throughout the aneurysms and to evaluate the amount of inflammation present due to the implanted SMP device and suture materials. Haematoxylin & Eosin (H&E) was used as a general stain to evaluate the overall amount of inflammatory cells and level of neovascularization that were present at the time points evaluated. Masson's Trichrome and Mallory's Phosphotunsic Acid Hematoxylin (PTAH) were the two stains used to evaluate the amount of elastic fibers and residual fibrin that were present throughout the aneurysms.

Haematoxylin & Eosin (H&E) is a Micro-atomic staining procedure that provides informative information about the general pathology of the tissue being evaluated. By performing an H&E stain on the tissues, a general overview of the type and amount of cells that are in an area can be determined due to the enhanced differentiation of nuclei with this stain. Eosin, is an acid dye that stains cytoplasm of RBCs and connective tissues various shades of red, pink and orange. The nuclei of cells are stained blue/black by Haematoxylin.

Masson's Trichrome is one of two connective tissue staining procedures that were used to evaluate the level of elastic fibers and residual fibrin throughout the aneurysms. Masson's Trichrome stain stains collagen and reticular fibers stain blue/green, cytoplasm and red blood cells stain red and the nuclei will be stained black/gray (Brown G G, Carnes W H. Primer of Histopathologic Technique: Meredith Corporation; 1969 and Putt F A. Manual of histopathological staining methods. 1972:335). Trichrome was used to determine the amount of collagen and elastic fibers at each time point. Mallory's Phosphotunsic Acid Hematoxylin (PTAH) is the second connective tissue staining procedure that was performed.

This stain was used to determine the amount of residual fibrin within the aneurysm dome. PTAH can also be used to determine the amount of collagen and elastin that has been deposited. These stain colors nuclei of cells a deep blue, fibrin fibers stain a lighter blue, collagen stains a reddish color, coarse elastin fibers stain bluish in color.

### Results:

Gross Evaluation of Healing

For zero days, the surgical site was clean, as expected for being within the body for less than twenty four hours and the polymer had visible thrombus throughout the polymer matrix where it had been in contact with blood flow. For the thirty day implants, the outer surface was composed of a white to slightly opaque dense connective tissue, which had tan to golden brown patchy discolorations. Two clips of the cranial end and the carotid artery was bisected to expose the on face view of the ostium of the aneurysm sac. The surface of the ostium was white, glistening and appeared to be intact. However, when viewed by the naked eye, there appeared to be focal areas where polymer was exposed. For implants that had remained in vivo for ninety days, the eternal surface of the aneurysms showed multifocal adhesions of dense white connective tissue with tan to golden brown discoloration. Similarly to the thirty day aneurysms, the carotid artery was bisected to visualize the ostium and it appeared to be white, glistening, intact and lacking exposed polymer.

Microscopic Evaluation of Healing

### LV-SEM: Endothelialization

For microscopic evaluation of endothelialization at the foam and parent artery interface. low vacuum scanning electron micrographs (LV-SEM) indicate that at zero days the porous surface that was in contact with the vessel lumen had patchy aggregates of fibrin-enmeshed red blood cells on the surface of the polymer implant. After thirty days there was a presence of a discontinuous endothelial layer due to a disruption by polymer struts. When the ostium was imaged en face there was an endothelial cell covering and that there was a mixture of cobble-stone patterned and spindle-shaped endothelial cells. These endothelial cells aligned parallel with the long axis of the parent vessel. Table 1 summarizes the topographical LV-SEM evaluation of all aneurysms.

TABLE 1
Summary of endothelial covering of the base of the aneurysms

| | | 10% and | >5% and | <5% | |
|---|---|---|---|---|---|
| | >20% of | <20% | <10% | incom- | 100% |
| Implan- | surface | of surface | of surface | pletely | covered |
| tation | incompletely | incompletely | incompletely | covered by | by |
| Time, | covered by | covered by | covered by | endo- | endo- |
| days | endothelium | endothelium | endothelium | thelium | thelium |
| 0 | 2 of 2 | | | | |
| 30 | — | 1 of 4 | 2 of 4 | 1 of 4 | |
| 90 | — | | | | 6 of 6 |

It was shown that after ninety days, all of the aneurysms were completely covered by endothelial cells aligned parallel with the long axis of the carotid artery.

Table 2 summarizes the remnants of mural thrombus at the base of the aneurysms evaluated via LV-SEM imaging.

TABLE 2
Summary of residual mural thrombus determined via LV-SEM imaging

| Implan- | >10% of | >5% and | >1% and | | |
|---|---|---|---|---|---|
| tation | surface | <10% | <5% | <1% | 0% covered |
| Time, | covered by | covered by | covered by | covered by | by |
| days | thrombus | thrombus | thrombus | thrombus | thrombus |
| 0 | 2 of 2 | — | | — | — |
| 30 | — | 4 of 7 | 1 of 7 | 2 of 7 | — |
| 90 | — | — | — | 6 of 6 | — |

It was shown that there was less than 1% mural thrombus remaining on the surface of the base of the aneurysm after thirty days, and no thrombus remaining after ninety days.

Pathology:

### Connective Tissue: Aneurysm Composition

Table 3 summarizes the composition of the connective tissue of each aneurysm at the three time points.

TABLE 3
Summary of aneurysm sac composition

| | 0% | 25% | 50% | 75% | 100% |
|---|---|---|---|---|---|
| Implan- | composed | composed | composed | composed | composed |
| tation | of | of | of | of | of |
| time, | connective | connective | connective | connective | connective |
| days | tissue | tissue | tissue | tissue | tissue |
| 0 | 2 of 2 | | — | — | — |
| 30 | — | 4 of 7 | 1 of 7 | 2 of 7 | — |
| 90 | — | — | — | 6 of 6 | — |

It was shown that at zero days there was no connective tissues present. At thirty days there were connective tissues present between 25-75% throughout all aneurysms. At ninety days there was 75% connective tissue present throughout the aneurisms.

Remnant Fibrin Present in Aneurysm

Table 4 summarizes the amount of remnant fibrin, or the amount of fibrin that had not been resolved, or fully degraded throughout the aneurysm domes at each time point.

TABLE 4
Summary of fibrin remaining in the aneurysm dome

| Implan- | | | | | | |
|---|---|---|---|---|---|---|
| tation | | | | | | |
| time, | >50% | >25% and | >10% and | <5% and | <5% | 0% |
| days | fibrin | <50% fibrin | <25% fibrin | <10% fibrin | fibrin | fibrin |
| 0 | — | — | — | 2 of 2 | — | — |
| 30 | — | — | 3 of 7 | 3 of 7 | | — |
| 90 | — | — | 1 of 6 | | 5 of 6 | — |

At the zero day time point, less than one hour exposure to blood prior to euthanization, there was between 5 and 10% fibrin present. After thirty days of implantation, one of seven aneurysms evaluated was composed of more than 50% fibrin, with the remaining six being composed of 5-25% fibrin. After ninety days, there was one aneurysm composed of between 10 and 25% fibrin, with the remaining five being composed of less than 5% fibrin.

Connective Tissue within the Aneurysm

Table 5 is a summary of the connective tissue within the aneurysms; it is expressed in the form of a percentage of complete infiltration with dense, cellular connective tissue of the aneurysms at each time point throughout the volume of the dome.

TABLE 5
Percentage of complete infiltration with dense, cellular connective
tissue: First number is representative of the pathology at the anastomosis
interface, the second number listed is representative of the pathology
of the inner core of the aneurysm, and the third number is the pathology
representative of the apex of the aneurysms.

| Implan- | | | | | | |
|---|---|---|---|---|---|---|
| tation | | | | | | |
| time, | Number of | | >75% and | >85% and | | |
| days | aneurysms | <75% | <95% | <95% | >95% | 100% |
| 0 | 2 | — | — | — | — | — |
| 30 | 7 | 0; 1; 4 | 0; 1; 0 | 3; 3; 1 | 4; 2; 2 | 0; 0; 0 |
| 90 | 7 | 0; 0; 0 | 0; 0; 0 | 1; 2; 1 | 4; 2; 2 | 0; 0; 0 |

There was no cellular infiltration at zero days. At thirty days, there was greater than 75% cellular infiltrates throughout the aneurysm volume. At ninety days, there was greater than 85% cellular infiltration in all aneurysms. Inflammation present within the aneurysm dome and vicinity of the aneurysm.

Table 6 is a summary of the general inflammatory response present within the dome of the aneurysms elicited by the foam determined by the average number of inflammatory cells present.

TABLE 6
Percentage of inflammation determined by the amount of inflammatory
cells present: First number is representative of the pathology at the
anastomosis interface, the second number listed is representative of
the pathology of the inner core of the aneurysm, and the third number
is the pathology representative of the apex of the aneurysms.

| Implan- | | | | | | |
|---|---|---|---|---|---|---|
| tation | | | | | | |
| time, | Number of | | >10% and | >5% and | | |
| days | aneurysms | >25% | <25% | <10% | <5% | 0% |
| 0 | 2 | — | | | | |
| 30 | 7 | 0; 0; 0 | 2; 0; 2 | 5; 6; 4 | 0; 1; 1 | 0; 0; 0 |
| 90 | 6 | 0; 0; 0 | 0; 0; 0 | 4; 0; 4 | 2; 6; 2 | 0; 0; 0 |

Inflammation induced by the presence of foam was evaluated at three positions throughout the volume.

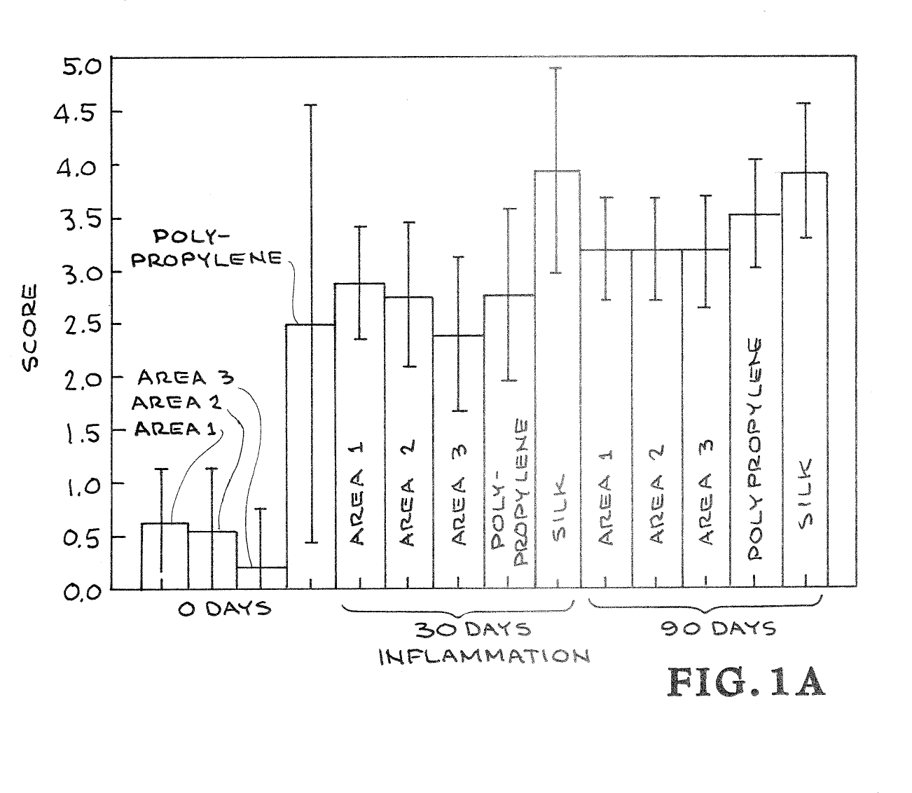

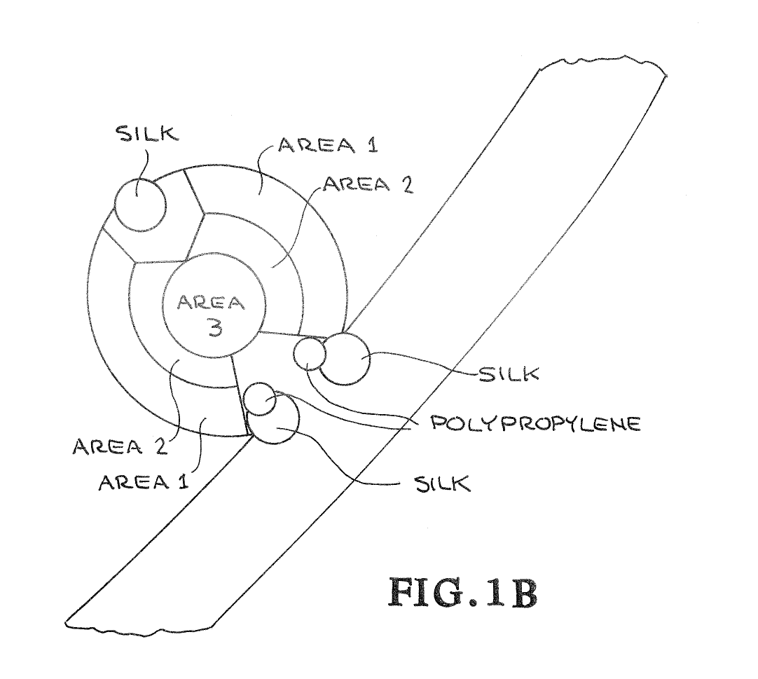

Referring to FIG. 1A and FIG. 1B, Area 1 represents the perimeter of the aneurysm minus the areas proximal to sutures. Area 2 represents the area between the peripheries and the core. Area 3 represents the core, or middle of the aneurysm domes. For zero days, essentially zero inflammatory cells were present due to the short in vivo exposure time; with on average less than one leukocyte present per 250× magnification area for the volume of foam evaluated. At thirty days, on average all aneurysms had less than 25% inflammatory cells present throughout the dome and approximately 4-6 leukocytes per 250× magnification area. At ninety days, all aneurysms had less than 10% inflammatory cells present and approximately 5-8 leukocytes per 250× magnification area.

As to the general inflammatory response present within the dome of the aneurysms elicited by the foam at zero days, polypropylene suture material was the only suture material evaluated do to a lack of silk incorporated into the tissue during histology. On average, it was shown that there was between three to eight leukocytes per 250× magnification area. At thirty days polypropylene suture material induced inflammation that corresponded to approximately four to eight leukocytes per 250× magnification area. For silk, it was shown that there were approximately eight to eleven leukocytes per 250× magnification area evaluated (thirty days). At ninety days it was shown that there were approximately five to eleven leukocytes per 250× magnification area for polypropylene sutures and approximately eight to eleven leukocytes per 250× magnification area evaluated. Additionally, there were focal mineralization observed proximal to the polypropylene sutures after thirty days and focal mineralization around the silk sutures after ninety days of implantation.

Visual comparison of polymer and suture material interaction can be seen in FIG. 1A and FIG. 1B (A.30 and A.90) for thirty and ninety day time points.

Inflammation around suture material was evident as illustrated in FIG. 1A and FIG. 1B (C.30 and C.90), which is represented by an abundance of multinucleated giant cells when compared to the polymer interaction in vivo—FIG. 1A and FIG. 1B (B.30 and B.90). In FIGS. 1A and 1B the notations are as follows: A.30) 2×H&E staining of a bisected aneurysm exhibiting the foam and suture interaction after thirty days of implantation, B.30) 8×H&E staining of a middle section of a bisected aneurysm after thirty days of implantation showing the foam-body interaction, C.30) 8×H&E staining of suture interaction after thirty days of implantation. Diamond indicates polypropylene suture and doughnut indicates focal mineralization adjacent to the polypropylene suture material, D.30) 8×H&E staining of suture interaction after thirty days of implantation. Diamond indicates silk suture A.90) 2×H&E staining of a bisected aneurysm after ninety days of implantation, B.90) 8×H&E staining of a middle section of a bisected aneurysm after ninety days of implantation showing the foam-body interaction, C.90) 8×H&E staining of suture interaction after ninety days of implantation. Diamond indicates polypropylene suture and doughnut indicates focal mineralization adjacent to the polypropylene suture material, D.90) 8×H&E staining of suture interaction after ninety days of implantation. Diamond indicates silk suture.

Healing present throughout the aneurysm dome based on neovascularization.

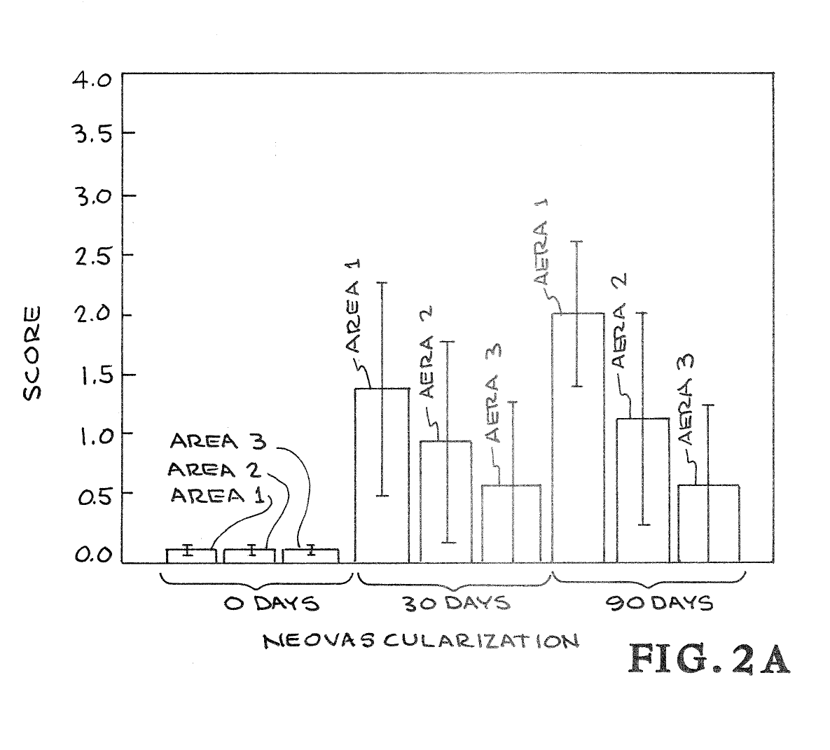

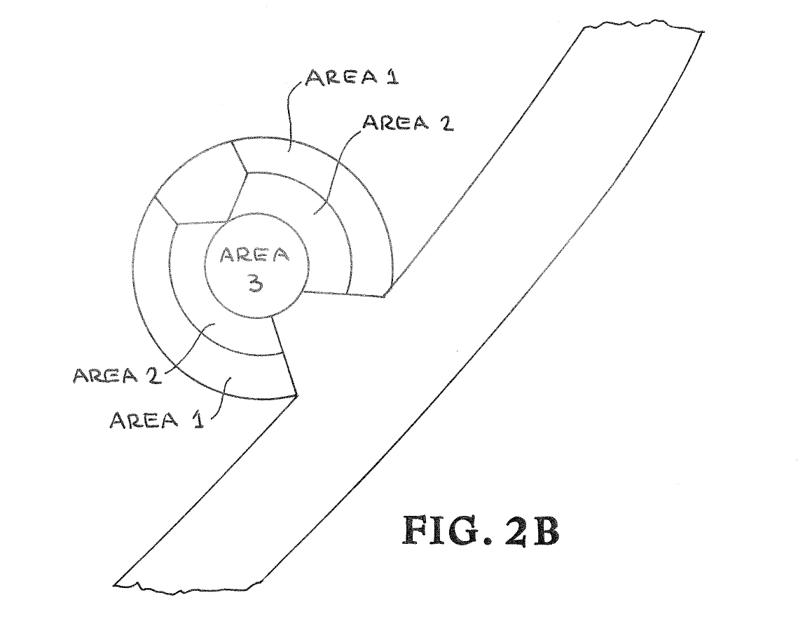

FIG. 2A and FIG. 2B summarize the amount healing throughout the aneurysm domes by quantifying the amount of neovascularization within the three areas evaluated. The notations are as follows: A score of zero indicated no of neovascular buds present per 250× magnification. A score of one indicated minimal or one neovascular bud per 250× magnification. A score of two indicated two to three neovascular buds per 250× magnification. A score of three indicated four to five neovascular buds per 250× magnification. A score of four indicated more than five neovascular buds per 250× magnification. Black square indicates neovascular bud.

In FIG. 2A and FIG. 2B Area 1 represents the perimeter of the aneurysm. Area 2 represents the area between the peripheries and the core. Area 3 represents the core, or middle of the aneurysm domes. It was shown that at zero days implanted there was no neovascularization, at thirty days implanted there was approximately one neovascular bud per 250× magnification area evaluated, and at ninety days there was approximately two neovascular buds per 250× magnification area evaluated.

Aneurysm Obliteration

Based on fluoroscopic imaging performed during implantation, LVLV-SEM, and histological evaluation via light microscopy there was complete filling with SMP foam of all aneurysms at all implantation times.

Neointima Proliferation at the Base of the Aneurysms.

Table 7 summarizes the amount of neointima proliferation at the base of the aneurysm that was determined via LV-SEM and light microscopy.

TABLE 7
Summary of neointima proliferation at the base of the aneurysms
determined by LV-SEM and light microscopy

| Implantation | >5% and <20% | <5% | 0% |
|---|---|---|---|
| time, | lumen | lumen | lumen |
| days | narrowing | narrowing | narrowing |
| 0 | — | — | 2 of 2 |
| 30 | 2 of 7 | 5 of 7 | — |
| 90 | — | 6 of 6 | |

It is shown that at zero days there was no narrowing of the parent vessel's neointima at the site of implantation. There was approximately 5% narrowing of the parent vessel at thirty days, and for two aneurysms, there was between 5 and 20% narrowing. At ninety days there was less than 5% narrowing of the parent vessel for all aneurysms.

### Discussion of Tests:

The purpose of above described test was to demonstrate that polyurethane based SMP foam of the invention is biocompatible in vivo when implanted into a porcine vein pouch aneurysm model. These devices were implanted in a range of time points between zero and ninety days. Integrative techniques (LV-SEM imaging and histology) were performed for all aneurysms. Histology verified that the SMP foams are biocompatible and was effective at providing a biological scaffold. This scaffolding enhanced the healing response as exhibited by the presence of predominant connective tissue substrate at ninety days.

It was also shown that the SMP foam material has a reduced inflammatory response when compared to conventional suture materials (monofilament polypropylene and braided silk). In 2005, Karaca et al. showed that both suture materials, silk and polypropylene, promote a granulomatous inflammation around the implant with varying severity (Karaca E, Hockenberger A S, Yildiz H. Investigating Changes in Mechanical Properties and Tissue Reaction of Silk, Polyester, Polyamide, and Polypropylene Sutures in Vivo. Textile Research Journal 2005 April 01; 75(4):297-303. Furthermore, Karaca et al. demonstrated that braided suture materials promoted more inflammation than monofilaments. Inflammation noted around these suture materials consisted of a “purulent core surrounded by inflammatory cells and an outer fibrous encapsulation”. These results coincide with the results reported by Chu et al. in 1997 (Chu C-, von Fraunhofer J A, Greiser H P. Wound closure biomaterials and devices. Boca Raton, Fla.: CRC Press; 1997) who noted that in less than one month silk and polypropylene elicit a marked and moderate reaction respectively. And when implanted for up to 24 months, silk and polypropylene, the materials used in the formation of the aneurysm sac, elicit moderate and slight reactions respectively, which correlated to the ninety day results evaluated in this study. The fibrous encapsulation of the foreign material is the hallmark of the end of inflammation elicited by a material. This encapsulation isolates the device/material from the tissue.

When an implanted material shows a reduced inflammatory response the connective tissue capsule concomitantly is less. When directly comparing the SMP foam to the braided silk and to some extent the polypropylene sutures, the perimeter of biological tissues surrounding our SMP foam shows a thinner capsule and showed less inflammation than the current sutures used in this procedure (FIG. 1A and FIG. 1B).

The SMP foam was less likely to induce a chronic-active inflammatory response when compared to the silk; additionally the SMP foam provided a scaffold for healing. Whether or not an implant leads to healing or sustained inflammatory inflammation is dependent upon host's response to the implanted material. Ideally, the host inflammatory response should be minimal to mild and of short duration.

Biomaterial Implants may elicit a sustained foreign body reaction for the life of the implant, but the severity of the reaction is dependent upon the physical and chemical properties of the biomaterials. These properties ultimately determine the intensity and duration of the host's immune response. A chronic or chronic-active inflammatory reaction can lead to impairment of the function of the tissue in which the implant is located. Alternatively, if inflammation elicited by the implant is ceased early it could be considered biocompatible. The reduced inflammatory response elicited by the SMP foam leads to an earlier transition toward healing; evident by the laying down of collagen and elastin throughout the dome of the aneurysm and a reduced population of multinucleated giant cells surrounding polymer struts. Transition to healing was also present in the areas where sutures were used, but when compared to the SMP foam there were more cellular inflammatory components.

Granulation tissue (early stage of healing) was present throughout the volume of the filled aneurysm dome. Granulation tissue was identified as the presence of collagen, neovascular buds and a small number of macrophages, and/or multinucleated giant cells; to a lesser extent were eosinophils and neutrophils. In the later stages of healing, collagen type III is predominantly present. This fibrous connective tissue substrate with minimal inflammatory cells is the final transition to host/biomaterial compatibility. In the aneurysms it was shown that the healing process occurred around all materials used, with SMP foam, polypropylene and silk being the rank of lowest inflammation to highest throughout the process.

Neovascular bud infiltration throughout the aneurysm device was also evaluated.

The presence of neovascularization indicates the active healing process. It was shown that by ninety days there was an increased number of neovascular buds. A previous study, comparing polyurethane based foams to metal coils, showed that intravascular implantation of the foams resulted in a faster and more safe occlusion method within the volume of foam (Kipshidze N, Sadzaglishvili K, Panarella M, Elias A. Rivera M, Virmani R, and Leon M B. Evaluation of a Novel Endoluminal Vascular Occlusion Device in a Porcine Model: Early and Late Follow-up. Journal of Endovascular Therapy 2005; 12(4):486-494). The interconnected pores of open celled foams encourage permeation of neovascular and cellular ingrowth.

The physical condition of the SMP foam implant lacked both compaction of foam and a residual neck. Previously it was shown by Cognard et al. in 1999 (Cognard C, Weill A, Spelle L, Piotin M, Castaings L, Rey A, et al. Long-term angiographic follow-up of 169 intracranial berry aneurysms occluded with detachable coils. Radiology 1999 August; 212(2):348-356), that there is a possibility for reoccurrence of aneurysm growth at the residual neck when the aneurysms had been treated by GDCs. Aneurysm regrowth occurred, due to compaction of coils over a period of time when the sac had been occluded at the point of treatment. In this study they demonstrated that within the time frame of 3-40 months after initial treatment recurrence of aneurysms was observed due to coil compaction. Although the foams of this test were implanted for more than three months, there was no compaction of foam shown throughout our study, and isolation from the vasculature of the aneurysm dome was complete at ninety days, which is evident by the endothelial cell lined fibrous cap at the aneurysm and parent artery interface and the complete neointima growth observed. Of note, there was no gross or microscopic evidence of active thrombogenesis over the endothelialzed neck.

It has been proposed that recurrence of aneurysm growth is facilitated by compaction of metal coils within the aneurysm sac due to the constant impingement of arterial blood flow. Compaction of metal coils would occur when a material had not promoted stabilization within the aneurysm fast enough to prevent aneurysm reformation, as shown by Szikora et al. in 2006; this lack of stabilization and healing is most likely due to the lack of biological activity of the platinum coils (Szikora I, Seifert P, Hanzely Z, Kulcsar Z, Berentei Z, Marosfoi M, et al. Histopathologic evaluation of aneurysms treated with Guglielmi detachable coils or matrix detachable microcoils. AJNR Am J Neuroradiol 2006 February; 27(2):283-288). In contrast to platinum, SMP foams initiate blood clotting within the aneurysm, in less than thirty minutes, promoting stabilization and healing throughout, thereby decreasing the possibility of recurrence of aneurysm formation.

These tests demonstrate that the SMP open cell foams of the invention are effective in inhibiting inflammation and in promoting healing in implanted devices in living tissue.

Implantable devices for treatment and sealing of vascular, septal and other bodily abnormalities that require blood clotting for effectiveness that are made from the open cell foam compositions described above will reduce inflammation and promote healing at the site of the implant. Such devices are within the scope of this invention.

While the invention may be susceptible to various modifications and alternative forms, specific embodiments have been shown by way of example in the drawing and have been described in detail herein. However, it should be understood that h the invention is not intended to be limited to the particular forms disclosed. Rather the invention is to cover all modifications, equivalents and alternative falling within the spirit and scope of the invention as defined by the following appended claims.

The invention claimed is:

1. A method of reducing inflammation and promoting healing in vascular embolic applications comprising implantation of an open cell polymer foam scaffold in a vascular abnormality to promote blood clotting and growth of extracellular matrix throughout the scaffold.

2. The method of claim 1 wherein the open cell polymer foam scaffold have cell sized from 50 nm to 5 mm and have at least 20% of membranes between cells removed.

3. The method of claim 2 wherein the open cell polymer scaffold have at least 50% of membranes between cells removed.

4. The method of claim 1 wherein the polymer is selected from the group consisting of polyurethanes, polyacrylates, polymethacrylates, polyolefins, polyorganosiloxanes, polyesters, polyethers, and copolymers of polymers.

5. The method of claim 1 wherein the open cell polymer foam is made from monomers of N,N,N′,N′-145 Tetrakis (2-hydroxypropyl) ethylenediamine (HPED), 2,2′,2″-Nitrilotriethanol (TEA) and 1,6 Diisocyanatohexane (HDI) and either chemical and physical blowing agents or both.

6. The method of claim 1 wherein the open cell polymer foam is biocompatible.

7. The method of claim 1 wherein the vascular abnormality is a cranial aneurism or septal opening.

8. A composition for use in implantable devices that reduce inflammation and promote healing comprising an open cell shape memory polymer foam with cells sized from 50 nm to 5 mm and have at least 20% of membranes between cells removed.

9. The composition of claim 8 wherein the open cell polymer scaffold have at least 50% of membranes between cells removed.

10. The composition of claim 8 wherein the polymer is selected from the group consisting of polyurethanes, polyacrylates, polymethacrylates, polyolefins, polyorganosiloxanes, polyesters, polyethers, and copolymers of polymers.

11. The composition of claim X wherein the open cell polymer foam is made from monomers of N,N,N′,N′-145 Tetrakis (2-hydroxypropyl)ethylenediamine (HPED), 2,2′,2″-Nitrilotriethanol (TEA) and 1,6 Diisocyanatohexane (HDI) and either chemical and physical blowing agents or both.

12. An implantable device or structure useful for treatment of vascular and septal abnormalities that require blood clotting for healing made from the composition of claim 8.

13. The device or structure of claim 12 made from the composition of claim 10.

14. The device or structure of claim 12 made from the composition of claim 11.
